(19) United States
(12) Patent Application Publication
(10) Pub. No.: US 2005/0131287 A1
(43) Pub. Date: Jun. 16, 2005

(54) Detection of premature rupture of the amniotic membrane

(75) Inventors: Rosann Marie Kaylor, Cumming, GA (US); RameshBabu Boga, Roswell, GA (US); Kaiyuan Yang, Cumming, GA (US); Shawn Ray Feaster, Duluth, GA (US); Curtis Neil Sayre, Atlanta, GA (US); David Samuel Cohen, Alpharetta, GA (US); Ning Wei, Roswell, GA (US); Chibueze Obinna U. Chidebelu-Eze, Atlanta, GA (US); Stephen Quirk, Alpharetta, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/736,662

(22) Filed: Dec. 16, 2003

(51) Int. Cl. A61B 5/00; B65D 81/00

(52) U.S. Cl. 600/362; 600/584; 600/309

(57) ABSTRACT

The premature rupture of amniotic fluid (PROM) may be discovered through a number of inventive means. Methods of evaluating whether PROM is present include; a) through the testing of the pH of vaginal fluids using an irreversible pH test; b) through the detection of analytes (e.g. enzymes) specific to amniotic fluid in the vaginal fluids; c) though the detection of hydrogen peroxide (H₂O₂) in the vaginal fluid; and d) through the detection of cholesterol in vaginal fluid. While individually indicative of PROM, it is desirable to combine at least two of these techniques to yield a powerful tool of even greater reliability. Test devices and feminine hygiene pads into which the test methods may be incorporated are also included herein.

### BACKGROUND OF THE INVENTION

Premature rupture of membranes (PROM) is defined as the rupture of the chorion/amnion membrane more than six hours prior to onset of childbirth contractions. When this occurs, amniotic fluid starts leaking, slowly or in a gush, into the vaginal canal. Without the normal functions afforded by amniotic fluid, e.g., protection against infection, protection against trauma, facilitation of free fetal movement, and preventing chord compression, continuation of the pregnancy places the fetus and mother at risk. Should this occur prior to the 37^{th} week of pregnancy, this is referred to as preterm premature rupture of membranes (pPROM) which instantly promotes a normal pregnancy to high risk status and represents a major source of perinatal morbidity. Although pPROM does not necessitate preterm delivery, the mother and fetus must be closely monitored for spontaneous onset of contractions, chorioamniontis, infection, heart rate, blood pressure, etc.

PROM is one of the most common complications during pregnancy. The reported incidence rates vary widely in different studies, most likely due to differences in demographics, study protocols, method of diagnosis in the study, etc. On average, however, it occurs in 10 percent of births of 37 weeks or more of gestation, and in 1-2 percent of births that occur before 37 weeks gestation. In about 10 percent of cases, regardless of gestation period, the fetus does not survive. The primary risk from PROM is infection to both the mother and fetus. The sooner that PROM is detected, therefore, the faster a physician can treat the patient, such as by the administration of antibiotics or by inducing labor.

One symptom of PROM that may occur is the discharge of liquid from the vaginal canal. This is often confused, however, with the normal vaginal secretions a woman may experience, which are not caused by PROM. Thus, there is a need for a reliable test to allow a woman and her physician to easily monitor her condition, and to be alerted if she is experiencing PROM.

Swab indicators suitable for in-home use for measuring the pH of vaginal moisture have often been used to test for the premature rupture of membrane. Users of such items may diagnose the early symptoms of PROM by inspecting the indicator for a pad color, without seeing a doctor. Unfortunately such pH based color change indicators are susceptible to a high rate of false positives or false negatives. The color change may be greatly affected by dilution by vaginal fluids, urine, and by contact, which often reverse the pH based color change.

Another method of detecting PROM is a test that consists of a reader with disposable test strips that is suitable for use in a physician's office. The test detects the presence of fetal fibronectin in a woman's vaginal secretions, associated with premature rupture of membrane (PROM) or the onset of birth. The test takes somewhat less than an hour for results. Physicians, however, prefer in-office tests to provide results within 15 minutes, so that they can determine the results while the patient is still in the exam room. Longer times for results require that the patient either remains in the waiting room for a long period of time or that the physician telephone them with the results.

Another commercial test measures the presence of estriol in saliva as a biomarker for pre-term birth and is designed as a mail-in test where a consumer may send her sample to a lab for analysis. This requires the user to wait for mail delivery and subsequent lab analysis and so is not suitable for symptomatic women or those suspecting premature rupture of membranes, since more immediate action must be taken.

The home use, office based, and mail-in formats described above lack simplicity and/or reliability and are not conducive to use by a non-professional, such as by a woman needing to monitor her condition without constantly having to visit a physician.

Previous methods have also had difficulty in distinguishing between preterm labor and premature rupture of the amniotic membrane. It is extremely important to distinguish between preterm labor and PROM because proper treatment is critical for the health of the infant and the mother.

### SUMMARY OF THE INVENTION

In response to the discussed difficulties and problems encountered in the prior art, new methods of evaluating whether PROM is present have been developed. These include testing of the pH of vaginal fluids using an irreversible pH test, detection of analytes (e.g. enzymes) specific to amniotic fluid in the vaginal fluids, detection of hydrogen peroxide (H₂O₂) in the vaginal fluid and the detection of cholesterol in vaginal fluid. It is desirable to combine at least two of these techniques to yield a powerful tool of even greater reliability. A third or fourth test may optionally be added.

The invention includes feminine hygiene pads as well as lateral flow and cell button devices having the inventive indicators present in a manner such that they will give an indication visible to the unaided eye in the presence of amniotic fluid.

### BRIEF DESCRIPTION OF THE DRAWINGS

FIG. 1 is a drawing of a feminine hygiene pad having a portion cut away.

FIG. 2 is a drawing of a cross-section of the feminine hygiene pad of FIG. 1 taken across the narrowest dimension of the pad.

FIG. 3 is a drawing of a test device which measures the pH and hydrogen peroxide concentration of a sample.

FIG. 4 is a drawing of a cell button device for the measurement of pH and hydrogen peroxide concentration.

### DETAILED DESCRIPTION OF THE INVENTION

The premature rupture of amniotic fluid may be discovered through a number of inventive means. Methods of evaluating whether PROM is present include; a) the testing of the pH of vaginal fluids using an irreversible pH test; b) the detection of analytes (e.g. enzymes) specific to amniotic fluid in the vaginal fluids; c) the detection of hydrogen peroxide (H₂O₂) in the vaginal fluid; and d) the detection of cholesterol in vaginal fluid. While individually indicative of PROM, it is desirable to combine at least two of these techniques to yield a powerful tool of even greater reliability.

Irreversible pH Detection Through Color Change

Normal vaginal pH varies between 3.5 and 5 during pregnancy. At the onset of PROM this pH rises to that of amniotic fluid; 7 to 7.5. Although pH is an acceptable means for establishing PROM, it is by no means definitive since other conditions can lead to an increased vaginal pH such as, for example, infection. In addition, a pH test using a reversible system may give ambivalent results in many cases because it may be reversed in the presence of urine, which is normally acidic. A pH based system should possess, therefore, an irreversible change that is induced at or around pH 6 or more desirably 7.

In one aspect of the invention, using color as the pH indicator, the reversible nature of the color change is avoided through the use of functionalized diacetylene lipids which are incorporated into liposomes using standard techniques. Upon exposure to UV light, the lipids cross-link via their diene functionality producing stable lipid vesicles.

The cross-linked liposomes undergo an irreversible hyperchromic spectral shift from blue (˜550 nm) to red (˜670 nm) in response to environmental pH. Without being bound to a particular theory, this transition is thought to stem from a repacking of the lipid chains from a local energy minima to a more global energy minima. Additionally, the pKₐ of shift can be adjusted to any desirable range by incorporating the appropriate functional group.

A suitable starting material for the practice of this aspect of the invention is 10,12-pentacosadiynoic acid derivatized with glutamic acid, producing a spectral shift at pH 6.5. Alternately, 3-(dimethylamino)propylamine can be used directly to produce a hypsochromic (blue to red) shift at pH 5.5. A combinatorial approach may be used to develop the optimum lipid precursor possessing the desired pKₐ and color.

Yet another aspect of this method of detecting PROM is through the use of an encapsulated dye. This may be achieved by, for example, encapsulating a pH insensitive chromophore or fluorogen dye within a pH sensitive encapsulating material with a pKₐ greater than 6.5 but less than 7.0. Any liquid that comes in contact with the capsules and possesses a pH greater than the pKₐ will induce an irreversible degradation of the encapsulating material, thereby causing the dye to leak and wick into the pad/tampon. Liquid having a pH below that of the pKₐ (e.g., normal vaginal secretions or urine) will not cause the degradation of the encapsulating material and therefore not cause a color change. The color of the encapsulating material should be chosen to mask that of the dye such that a distinct and visually prevalent color change is observed.

These materials are biocompatible and can easily be incorporated into a feminine hygiene product such as, for example, a pad or tampon type device to facilitate sampling as described below. If a pad, for example, is impregnated with pH sensitive liposomes or pH sensitive capsules, then any liquid entering the pad with a pH greater than the pKₐ of the liposomes or capsules will induce an irreversible color change, indicating the potential presence of amniotic fluid.

Irreversible pH Detection Through Formation of Hydrogel

Another method of detecting the change in pH indicative of amniotic fluid in the vaginal canal is through the formation of a hydrogel. This aspect of the invention uses polymeric materials which can exhibit very large swelling at the pH of amniotic fluid. Polyacrylic acid, polymethacrylic acid or poly acrylic acid and ethylene glycol copolymer, are suitable examples of such hydrogels.

Hydrogels exhibiting pH dependent swelling can be swollen from ionic networks, containing either acidic or basic pendant groups. In aqueous media, at appropriate pH and ionic strength, the pendant groups can be ionized, developing charges on the gel. The resulting electrostatic repulsions will greatly increase the uptake of the fluid in the network. In the gels, the ionization usually occurs in a media where the pH in the environment is above the pKa of the ionizable species of the pendant groups. In the polymers mentioned above, the pKa is around 4.3, and when the pH reaches this level, the ionization will gradually start. Chemical modification of these polymers, such as increasing crosslinking and hydrogen bonding component in the network, will increase the dissolution pH for the hydrogel. This way, the hydrogel can exhibit dramatic swelling when the amniotic fluid leaks.

A pH responsive hydrogel will build pressure due to material expansion upon exposure to elevated pH that will be felt in the vaginal area, thus signaling possible PROM.

Analyte Detection

Another method of detecting PROM is to detect analytes present in amniotic fluid and signal their presence. This may be done by incorporating an analyte-sensitive material into an encapsulating material that may be used to contain a dye. The encapsulating material could also be a substrate specific for an enzyme found preferably solely within amniotic fluid such as lysozyme, which is capable of hydrolyzing cellulose. Alternatively, the encapsulating material could be composed of polydiamines which can be degraded by diamino oxidase found in amniotic fluid. An antigen(s) or the like found specifically within amniotic fluid may be used to degrade, either by binding events or by catalytically destroying, the encapsulating material, resulting in a release of the encapsulated material. The encapsulated material may again be a pH insensitive dye that is distinct and easily detected by the unaided eye. The overall product is suitable for both pad and tampon type devices.

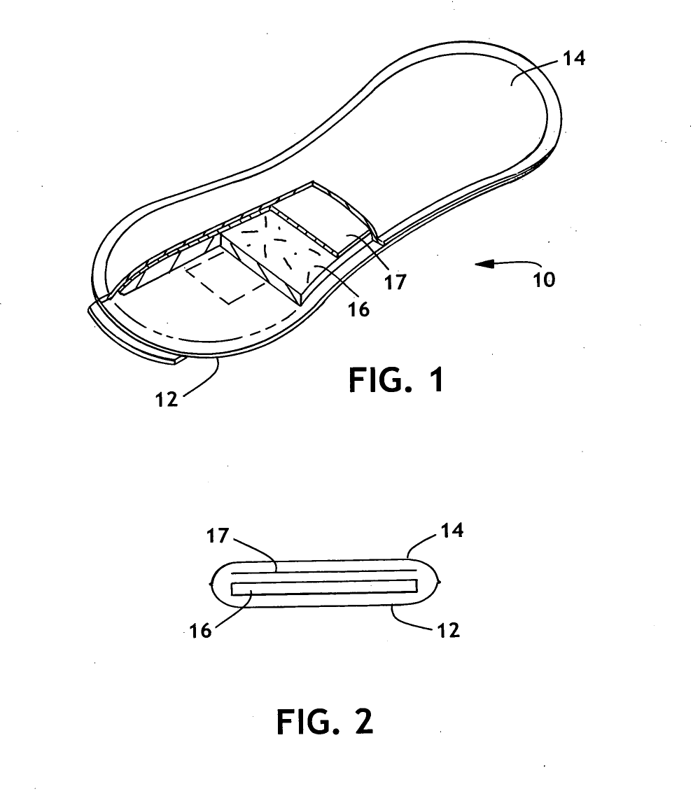

FIG. 1 shows a typical feminine hygiene pad partially cut away. This pad 10 has a liquid impervious baffle 12 on the side away from the wearer. The baffle 12 is often made from a film like a polyethylene or polypropylene film. The layer closest to the wearer is the liner 14 and is a liquid permeable layer that is preferably soft and absorbent. Between the baffle 12 and liner 14 there may be a number of layers for different purposes, such as an absorbent core 16 designed to hold the majority of any liquid discharge. Other optional layers include a transfer delay layer 17, and tissue wraps (not shown). The analyte-sensitive capsule containing dye may be placed upon the liner of the pad in the “target area”; the area normally contacted by vaginal discharges.

In another aspect of the invention a ligand receptor for a protein or analyte specific to amniotic fluid, such as lysozyme, diamino oxidase, or pulmonary surfactant protein may be deposited within a pad or tampon. A secondary receptor specific for an alternate site on the analyte may be deposited in a predefined pattern in another area or layer of the pad/tampon. The fluid entering the pad/tampon may be channeled to the ligand receptor deposit and then to the predefined receptor pattern, resulting in a visual indication of the presence of the analyte. FIG. 2 is a drawing of a cross-section of the feminine hygiene pad of FIG. 1 taken across the shortest dimension of the pad. The ligand receptor may be deposited on the liner 14 in the target area and the receptor on the layer below the liner 14, in this case the transfer delay layer 17, in the target area. The receptor is still visible through the liner 14 since the liner 14 is quite thin.

Biomarker analytes, especially those that would be unique to amniotic fluid in relation to urine and vaginal fluid include, but are not limited to alkaline phosphatase, diamine oxidase, monoamine oxidase, pepsinogen, alpha-galactosidase, alpha-fucosidase, amylase, alpha-mannosidase, and other carbohydrate-based enzymes, lysozyme, phosphatidic acid, phosphohydrolase, fetal fibronectin, alpha fetoprotein, collagen breakdown products, estradiol (also seen in saliva prior to onset of birth), active ceruloplasmin, aderenomedullin, insulin-like growth factor-binding protein, inhibin B, human chorionic gonadotropin, human placental lactogen, granulocyte elastase, prolactin, fructose-based fatty acids, lipids (e.g., phospholipids, lecithin), uric acid, urea, creatinine (may also be in urine), renin.

Of these, the enzymes present in amniotic fluid are a desirable way to obtain a secondary chemical reaction that could be easily indicated and detected by a user. Specific enzymes in amniotic fluid at certain times during gestation as well as their detection methods include the following:

Alkaline phosphatase (ALP) has been reported at levels of 27.2±11.9 mU/mL in amniotic fluid at third trimester (Geyer, V. H. in Die Herkunft der Furchtwasser-Enzyme, Z. Klin. Chem., 8, 145 (1970)). Note that ALP is also present in blood in some conditions, but has not been reported to be in urine. Alkaline phosphatase may be detected using p-Nitrophenyl phosphate, di-sodium salt which yields a yellow color (405 nm) and which is commercially available from Kirkegaard and Perry Labs (Gaithersbury, Md., USA), catalog number 50-80-00 or 50-80-01 having a detection limit to 10⁻¹³ moles of alkaline phosphatase. Alkaline phosphatase may also be detected using 5-bromo,4-chloro,3-indolylphosphate(BCIP)/nitroblue tetrazolium (NBT), which yield a purple/black precipitate. Alkaline phosphatase is commercially available from Kirkegaard and Perry Labs, catalog number 50-81-18, and has a detection limit to 1 ng.

Diamine oxidase has been reported at levels of 17092±809 U/mL in amniotic fluid at third trimester (Southren et al., in J. Appl. Physiol., 20, 1048 (1965)) and may be detected using a hydrogen peroxide dependent substrate (e.g., diaminobenzidine tetrachloride or 3,3′,5,5′-tetramethylbenzidine (TMB)) with polyamine or other amine-containing polymer. The hydrogen peroxide generated from the reaction of the amine-containing polymer and diamine oxidase, if present, will then cause the substrate to form a colored material. Both of the substrates mentioned above are commercially available from Kirkegaard and Perry Labs.

Alpha-Galactosidase was reported at levels of 0.006 to 0.016 (±0.011) nmoles 4-methyl-umbelliferone/min/mL in second and third trimesters (Butterworth, J., et al., in Amer. J. Obstet. Gynec., 119, 821 (1974)). Beta-Galactosidase was reported at levels of 0.022 to 0.029 (±0.018) nmoles 4-methyl-umbelliferone/min/mL in second and third trimesters (id). Detection methods include the following exemplary substrates:
o-Nitrophenyl-β-D-galactopyranoside which produces a yellow color (405 nm) and is available from Sigma-Aldrich of Milwaukee, Wis., USA.
Naphthol-AS-BI-β-D-galactopyranoside.
4-Methyl-umbelliferyl-β-D-galactopyranoside, commercially available from Sigma-Aldrich.
5-Bromo-4-chloro-3-indolyl-β-D-galactopyranoside, available from Blotium, Inc. as “X-Gal” and which yields a blue precipitate.
5-Iodo-3-indolyl-beta-D-galactopyranoside, commercially available from Blotium, Inc.
N-methylindolyl-beta-D-galactopyranoside, commercially available from Blotium, Inc.
5-bromo-6-chloro-3-indolyl-beta-D-galactopyranoside, commercially available from Blotium, Inc.
6-chloro-3-indoxyl-b-D-galactopyranoside, commercially available from Blotium, Inc.

Alpha-Fucosidase was reported at levels of 1.05 (±0.5) nmoles p-nitrophenol/min/mL in the second and third trimesters (Butterworth, J., et al., in Amer. J. Obstet. Gynec., 119, 821 (1974)) and may be detected by colorimetric methods using 4-nitrophenyl-a-L-fucopyranoside (available from Sigma-Aldrich) as the substrate and which yields a yellow color (405 nm).

Amylase was reported at levels of 56.0±49.1 mU/mL in amniotic fluid at the third trimester (Geyer, V. H. in Die Herkunft der Furchtwasser-Enzyme, Z. Klin. Chem., 8, 145 (1970)). Note that amylase is also present in blood and urine (Clinical Guide to Laboratory Tests, 3^{rd} edition, 1995, ed. Norbert Tietz, ISBN 0-7216-5035-X) though amylase is not stable in acidic urine and so the environment surrounding the substrate should be controlled to a neutral to basic pH in order to ensure that only amylase from amniotic fluid is detected. Amylase may be detected using colorimetric methods using 4,6-Ethylidene-p-nitrophenyl-a-D-maltoheptaside as substrate which yields a yellow color (405 nm).

The amount of substrate used to detect the PROM-specific enzymes can vary. A useful parameter to determine the appropriate amount is the Michaelis constant (Kₘ), which is known to those skilled in the art.

Yet another aspect of the invention is to signal the presence of amniotic fluid analytes through the formation (or destruction) of highly conjugated segments on a polymer backbone. The formation of conjugated systems is preferred, because the change in going from non-colored to highly colored is more readily detected than in going from highly colored to non-colored. Development of such a conjugated system should be readily detectable.

Generation of the following type of polymer in situ should readily decarbonylate under mildly acidic conditions forming the unsaturated polymer, CO₂, and CO.

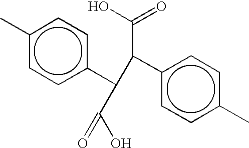

This assumes that the carboxylic acid groups were generated via enzymatic cleavage of an appropriate ester like galactoside and that the system was buffered. Again, a small amount of conjugation would give rise to a noticeable coloration, so small molecules (dye precursors) and low molecular weight polymers could also be used.

In yet another aspect of the invention, amniotic fluid may be detected enzymatically by placing, for example, 2,4-dinitrophenylhydrazine on a pad, along with the galactoside acetal of a ketone-containing polymer, such as poly(ester-ether-ketone) or PEEK. In the presence of amniotic fluid, the acetal is enzymatically hydrolyzed, and the ketone is liberated. A buffer is required so that acidic conditions do not hydrolyze the acetal and release the carbonyl. The ketone will then react with the hydrazine to form the hydrazone, which is a bright yellow or orange precipitate. Any other hydrazine compound that, when it reacts with carbonyl compounds, produces a colored product that is visible to the unaided eye may also be used, e.g., phenylhydrazine, nitrophenylhydrazine and the like.

Hydrogen Peroxide Detection

Hydrogen peroxide detection may be accomplished with hydrogen peroxide-mediated enzymatic and non-enzymatic conversion of chromophores. The chromophore can be a colorometric-, fluorescent-, or chemi-luminescent-based reagent. If the chromophore is placed on the liner 14 in FIG. 1, for example, the amniotic fluid will interact with the peroxidase substrates (e.g., tetramethylbenzidine (TMB) or o-phenylenediamine (OPD)), which react with hydrogen peroxide to give an indicative color for the presence of hydrogen peroxide.

Cholesterol Detection

The list of biochemical markers that exists in amniotic fluid but not in vaginal secretions, urine or blood is not long. One of the more promising molecules on that list, however, is cholesterol. A series of enzyme-based reactions may be employed to detect the presence of cholesterol.

Reaction for Cholesterol Measurement

| | | |
|---|---|---|
| Cholesterol ester + H₂O | cholesterol ester- | cholesterol + fatty acid |
| | ase ----> | |
| Cholesterol + O₂ + H₂O | cholesterol | cholesten-3-one + H₂O₂ |
| | oxidase ----> | |
| 2H₂O₂ + phenol + 4- | peroxidase ----> | 4-benzoquinone- |
| aminoantipyrine | | monoimino- |
| | | phenazone + 4H₂O |

Cholesterol is most likely found only in the cell debris in vaginal secretions, not in the liquid fraction. The level of cholesterol in urine is likewise negligible. Cholesterol is, however, naturally found in amniotic fluid in the 20-100 mg/L concentration range. Contamination from blood will give a false positive reading but in the case of blood spotting it is advisable to consult a physician.

The final step in the reaction described above results in a blue color. A test for the presence of cholesterol on a liner, pad or vaginal swab, therefore, would be a useful indicator of amniotic fluid leakage. Turning again to FIG. 1, the liner 14 of the pad 10 may be coated with an indicator such as 4-aminoantipyrine. In the presence of vaginal fluid containing amniotic fluid and therefore cholesterol, the pad will turn blue, alerting the wearer of the possibility of PROM. Since cell debris from normal vaginal fluid could interfere with the test, it may be desirable to place the indicator below the liner and use a liner with a sufficiently small pore size and/or adequate basis weight to prevent cell debris from getting through the liner and contacting the indicator.

The methods of PROM detection discussed thus far may be combined in various combinations to yield a highly specific test for PROM. The following are examples of such combinations though they are by no means exhaustive.

Irreversible pH Detection with Analyte Detection

In another aspect of the invention, cross-linked liposomes discussed above may incorporate a ligand receptor for a protein or analyte specific to amniotic fluid, such as lysozyme, diamino oxidase, or pulmonary surfactant protein. Within the pad or tampon, a secondary receptor specific for an alternate site on the analyte may be deposited in a predefined pattern, and the cross-linked liposomes are concentrated in a different area or layer of the pad/tampon. The fluid entering the pad/tampon may be channeled to the liposome deposit then to the predefined receptor pattern, resulting in a visual indication of the presence of the analyte. In this case, if the pH is elevated and the analyte is present, then the liposomes experience an irreversible spectral transition and the analytes bind to the predefined pattern, resulting in a visual indication of a positive result. If, however, the analyte is not present and the pH is elevated, then the analyte signal will not be present in the predefined receptor pattern demonstrating the absence of amniotic fluid and that the pH is elevated from a cause unrelated to PROM. Finally, if the analyte is present but the pH is not elevated, then the predefined pattern will result, however, the color will have not changed, suggesting the early stages of PROM.

In yet another aspect of the invention, a pH transition within the range of amniotic fluid is used to trigger the release of a dye specific to an analyte in amniotic fluid. The pH sensitive encapsulating material with a pKₐ greater than 6.5 but less than 7.0 as described above may be used for the capsule. If the pH of the fluid is elevated, the capsule will degrade and the analyte sensitive dye will be released. If the analyte is present, then a color change will result. If the enzyme is not present then no color change will occur. One example of this aspect of the invention is to use cellulose acetate phthalate (CAP) to encapsulate the analyte-sensitive dye.

In another aspect of the invention, an encapsulating material composed of a material that is sensitive to the binding of an analyte as described above may contain a pH sensitive dye. If the analyte is present, the capsule will degrade and the pH sensitive dye will be released. If the pH is elevated, the dye will change color to provide a visual indication of PROM. PH sensitive dyes include nitrazine, bromothymol blue, phenolphthalein, etc.

In anther aspect of the invention, it is noted that most pH indicators involve a protonation/deprotonation reaction, usually of phenolic groups. If the phenolic groups were coupled to an alpha-galactoside, the indicator couldn't change configuration/color until the galactoside was removed. If the alpha-galactosidase were only present in amniotic fluid, then the color change would be a much more reliable indicator of PROM than the color change of a pH indicator alone.

The structure below, for example, shows phenolphthalein, a common acid-base indicator. In the presence of base, the phenol(s) are deprotonated, which allows the phenol to adopt a quinoid-like structure, and “pops” open the lactone. The formation of the conjugated system (the quinoid-like one) changes the color of the indicator. Attaching an alpha-galactoside unit to the phenols would prevent deprotonation/rearrangement until cleaved.

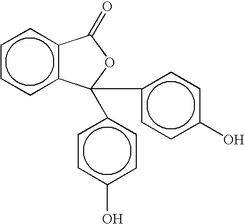

Nitrazene, for example, has three places that could be blocked with an alpha-galactoside, forming a desirable detection scheme for amniotic fluid. This type of indictor has the benefits of adjusting the pKa of the indicator to match that desired. The molar absorptivity can be selected (obviously for low level detections, it should be as large as possible), and the wavelength of response can also be tailored to that desired. By way of example; bromothymol blue has a pKa similar to nitrazene (7.00-7.30) and has a molar extinction coefficient of 3.75×10⁴ in the deprotonated form. Another example using fluorescence is the compound 4-methylumbelliferyl-alpha-D-galactoside which is commercially available and does not fluoresce while linked to the galactoside. Only when the galactoside is cleaved does it fluoresce. This can be used to detect PROM by fluorescence.

Similarly, a nitrazene analog as shown below could be constructed in situ through an enzymatic process. The imine linkage shown in the reaction below should spontaneously form and because of conjugation should be stable, even in the presence of water. If the aldehyde were protected as the acetal of galactose (and the device buffered at a slightly basic pH), it would not be available to form the imine until it was cleaved enzymatically. The enzyme would then have to effectively remove only one sugar, as the hemiacetal should readily convert back to the aldehyde.

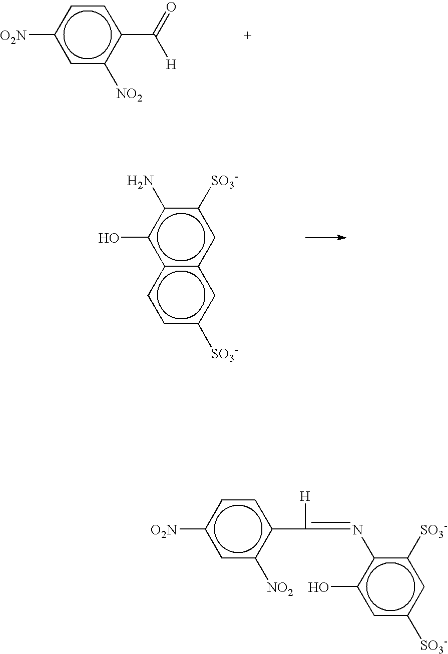

Another aspect to the detecting of amniotic fluid in the presence of urine would be to encapsulate the nitrazene (or other indicator) in a polysaccharide shell that would only be opened (thus exposing the indicator to the fluids) upon enzymolysis or hydrolysis of the encapsulant. For example, a pH sensitive gel (which limits diffusion) can contain the galactoside-blocked indicator. Under the proper pH conditions, the gel will shrink, expelling the galactoside-blocked indicator and exposing it to enzymes, if present.

Color generation need not be limited to an indicator or dye. The simple sugar dihydroxyacetone (DHA) combines with an amine to form an N-glycosamine via the Maillard reaction. DHA is most commonly found in self-tanning cosmetics. Blocking the hydroxyl groups with an alpha-galactoside unit prevents the Maillard reaction from taking place until the hydroxyl groups are freed. Spiking the device with a simple, non-volatile amine or amino acid would develop a brown color (much like a self-tanning cream would produce) upon release of the DHA.

Analyte Detection with Hydrogel Formation

Another option for the detection of PROM is to utilize the enzyme-based reaction to alter, only in the presence of amniotic fluid, the physical properties of a polymer, most notably absorbance. Two methods to bring about this type of change are to change binding to metal ions, or to change the conjugation of the backbone, both of which could produce a color change. Of these, modifications to the polymer backbone or sidechains which alter the absorptivitiy are simpler to implement, less toxic, and easier to detect by the unaided eye.

An ester may be made between galactoside and poly(acrylic acid) or a variant such as poly(methacrylic acid). A buffer should be present so that premature hydrolysis of the ester is minimized. This material should not swell when exposed to water. Upon hydrolysis by an enzyme in amniotic fluid, the free acid would be generated, and it would absorb (and be swollen by) any fluid present.

The esterified polymer may be applied in a pattern on, for example, the pad 10 of FIG. 1. In the presence of amniotic fluid, the pattern would raise/emboss, making the visual detection of amniotic fluid possible. This sort of “gel” may also be formed using galactoside prepared using polyvinyl alcohol (PVA) and having borate ions present.

pH and Hydrogen Peroxide Detection

This aspect of the present invention provides direct evidence of the changes in the vagina based on the pH and hydrogen peroxide detection, which are directly linked to the physiological status of vagina. It is possible for pH to vary irrespective of changes or a lack of changes in hydrogen peroxide levels, whereas hydrogen peroxide levels can definitively indicate the physiological status of the vagina and related infectious diseases. In other words, hydrogen peroxide can be a reliable bio-indicator when compared to the pH in vagina and related diseases. It can be a valuable tool to measure or detect both pH and hydrogen peroxide in vaginal fluids at any given time.

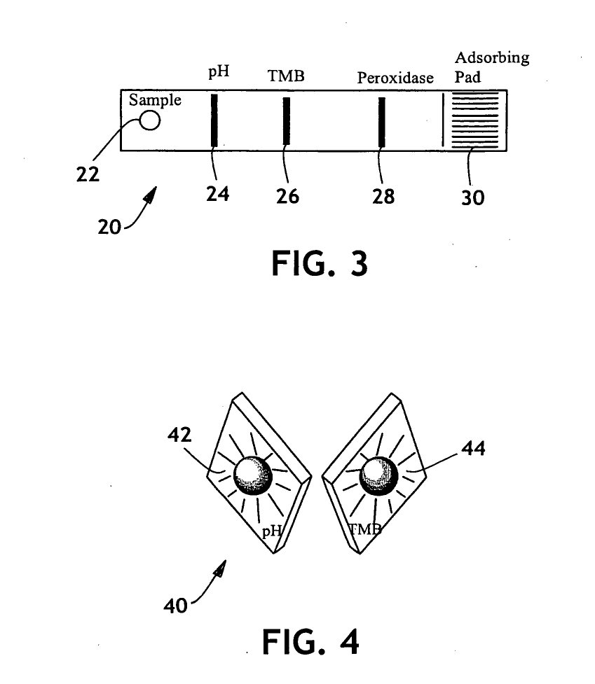

FIG. 3 shows a lateral flow device 20. In use, a sample will be deposited at a sample deposition point 22 and move towards a pH indicator 24 by capillary action, where the pH of the sample is measured. The sample will then interact with peroxidase substrates (e.g., tetramethylbenzidine (TMB) or o-phenylenediamine (OPD)) at a hydrogen peroxide test point 26, which will react with hydrogen peroxide to give an indicative color. This hydrogen peroxide-mediated conversion of chromophores can be carried out either enzymatically or non-enzymatically. The lateral flow device 20 also has a control peroxidase substrate 28 where the original peroxidase substrate will remain unchanged and an absorbing pad 30 to induce capillary flow.

In FIG. 4, a drawing of a cell button device is shown with the sides separated for ease of viewing. This device 40 can detect both pH and hydrogen peroxide by the vertical flow of a sample. As a sample is introduced on the pH side 42, it will indicate the pH, and then the sample will pass vertically to the peroxidase substrate side 44 to give a color characteristic of the presence of hydrogen peroxide. In normal vaginal conditions, therefore, the device 40 will show a color indicative of acidic conditions (e.g., red) on the pH side 42 because of the acidic nature of the vaginal fluid, and a blue color on the peroxidase substrate side 44 for the normal presence of hydrogen peroxide. A sample of vaginal fluid can thus be measured for both pH and hydrogen peroxide in a single device.

It has been shown above, therefore, that there are a myriad of possible two-pronged PROM detection methods possible through the eclectic choice of the many individual methods taught herein.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

1. A method of detecting the premature rupture of amniotic membrane comprising testing vaginal fluid for pH and determining a result as an irreversible change in a testing medium.

2. The method of claim 1 wherein said irreversible change is a color change.

3. The method of claim 1 wherein said irreversible change is production of a hydrogel.

4. The method of claim 2 wherein said testing for pH is performed using liposomes that undergo an irreversible hyperchromic spectral shift in response to an elevated environmental pH.

5. The method of claim 4 wherein said elevated pH is a pH of at least 6.

6. The method of claim 4 wherein said elevated pH is a pH of at least 7.

7. The method of claim 4 wherein said liposomes are selected from the group consisting of 10,12-pentacosadiynoic acid derivatized with glutamic acid, and 3-(dimethylamino)propylamine.

8. The method of claim 2 wherein said testing is performed using the steps of encapsulating a pH insensitive dye with a pH sensitive encapsulating material.

9. A method of detecting the premature rupture of amniotic membrane, comprising:
a first step selected from the group consisting of testing vaginal fluid pH and determining a result as an irreversible change, detecting amniotic fluid analyte in vaginal fluid, detecting hydrogen peroxide in vaginal fluid, and detecting cholesterol in vaginal fluid; and,
a second step, different from said first step, and selected from the group consisting of testing vaginal fluid pH and determining a result as an irreversible change, detecting amniotic fluid analyte in vaginal fluid, detecting hydrogen peroxide in vaginal fluid, and detecting cholesterol in vaginal fluid, wherein said first and second steps indicate premature rupture of amniotic membrane.

10. The method of claim 9 further comprising a third step, different from the first and second steps, and selected from the group consisting of testing vaginal fluid pH and determining a result as an irreversible change, detecting amniotic fluid analyte in vaginal fluid, detecting hydrogen peroxide in vaginal fluid, and detecting cholesterol in vaginal fluid.

11. The method of claim 9 wherein one of said steps is detecting amniotic fluid analyte in vaginal fluid, which results in a color change.

12. The method of claim 11 wherein said analyte is chosen from the group consisting of alkaline phosphatase, diamine oxidase, monoamine oxidase, pepsinogen, alpha-galactosidase, alpha-fucosidase, amylase, alpha-mannosidase, lysozyme, phosphatidic acid, phosphohydrolase, fetal fibronectin, alpha fetoprotein, collagen breakdown pads, estradiol, active ceruloplasmin, aderenornedullin, insulin-like growth factor-binding protein inhibin B, human chorionic gonadotropin, human placental lactogen, granulocyte elastase, prolactin, fructose-based fatty acids, phospholipids, lecithin, uric acid, urea, creatinine and rennin.

13. The method of claim 9 wherein one of said steps is detecting hydrogen peroxide in vaginal fluid and said detecting of hydrogen peroxide results in a color change.

14. The method of claim 13 wherein said color change is produced by a reaction between said hydrogen peroxide and a peroxidase substrate.

15. The method of claim 9 wherein one of said steps is detecting cholesterol in vaginal fluid and said detecting of cholesterol results in a color change.

16. The method of claim 15 wherein said color change is produced by a series of enzyme-based reactions including 4-aminoantipyrine.

17. The method of claim 9 comprising detecting amniotic fluid analyte in vaginal fluid by depositing a ligand receptor for an analyte in a first area of a feminine hygiene pad,
depositing a receptor specific to an alternate site on the analyte in a second area of said pad, and;
testing for pH by depositing cross-linked liposomes in a third area of said pad,
wherein fluid entering said pad is channeled to the ligand receptor deposit, then to the alternate site receptor and then to the third area of said pad, resulting in a visual indication of PROM.

18. The method of claim 9 comprising encapsulating an analyte sensitive dye within a capsule made from a pH sensitive encapsulating material with a pKa greater than 6.5 and less than 7, wherein said capsule releases said analyte sensitive dye and said dye changes color in amniotic fluid.

19. The method of claim 9 comprising encapsulating a pH sensitive dye within a capsule made from an analyte sensitive encapsulating material, wherein said capsule releases said pH sensitive dye and said dye changes color in amniotic fluid.

20. The method of claim 9 comprising coupling alpha-galactoside to phenolic groups of a phenolphthalein, wherein said alpha-galactosidase is cleaved and the phenolphthalein changes color in amniotic fluid.

21. The method of claim 20 further comprising the step of encapsulating alpha-galactosidase coupled phenolphthalein within an analyte sensitive capsule.

22. A feminine hygiene article comprising indicators selected from the group consisting of pH sensitive liposomes and pH sensitive capsules having a pH insensitive dye within.

23. A feminine hygiene article comprising a ligand receptor for an analyte deposited in a first area of the article and a receptor specific to an alternate site on the analyte in a second area of said article, wherein fluid entering said article is channeled to the ligand receptor deposit and then to the alternate site receptor, resulting in a visual indication of said analyte.

24. The article of claim 23 wherein said analyte is selected from the group consisting of alkaline phosphatase, diamine oxidase, monoamine oxidase, pepsinogen, alpha-galactosidase, alpha-fucosidase, amylase, alpha-mannosidase, carbohydrate-based enzymes, lysozyme, phosohatidic acid, phosphohydrolase, fetal fibronectin, alpha fetoprotein, collagen breakdown articles, estradiol, active ceruloplasmin, aderenomedullin, insulin-like growth factor-binding protein, inhibin B, human chorionic gonadotropin, human placental lactogen, granulocyte, elastase, prolactin, fructose-based fatty acids, lipids, uric acid, urea, creatine, and renin.

25. The article of claim 23 comprising a hydrazine and a galactoside acetal of a ketone-containing polymer, and a buffer, wherein said acetal is enzymatically hydrolyzed by amniotic fluid, and the ketone is liberated to react with the hydrazine to form hydrazone.

26. A lateral flow test for the detection of PROM comprising liposomes that undergo an irreversible hyperchromic spectral shift in response to an elevated environmental pH in first location on said lateral flow test and a peroxidase substrate in a second location on said test, wherein a sample of fluid passes through said first and second locations by capillary action.

27. A cell button device having a pH side and a peroxide side, wherein a sample of fluid introduced on the pH side will indicate a pH and then pass to the peroxide side and indicate peroxide.
